(19) United States
(12) Patent Application Publication
(10) Pub. No.: US 2008/0249458 A1
(43) Pub. Date: Oct. 9, 2008

(54) Intraventricular Shunt and Methods of Use Therefor

(75) Inventor: Dwayne S. Yamasaki, St. Augustine, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/058,262

(22) Filed: Mar. 28, 2008

Related U.S. Application Data
(60) Provisional application No. 60/910,768, filed on Apr. 9, 2007.

(51) Int. Cl. A61M 5/00

(52) U.S. Cl. 604/8

(57) ABSTRACT

Methods and apparatus for treating hydrocephalus caused by obstructions in the ventricular system and subarachnoid space of the brain. Embodiments include medical devices and methods for use within the subarachnoid space of the central nervous system to gain access to and treat an obstruction within the ventricles of the brain or a subarachnoid hemorrhage. The obstruction may be aspirated by an aspiration catheter for use in the subarachnoid space. A subarachnoid delivery catheter may then be used to deliver an intraventricular shunt to the opening in the obstruction via the subarachnoid space. A distal end of the delivery catheter is navigated across the obstruction to deploy the intraventricular shunt therein. The intraventricular shunt so positioned reopens the pathway between the ventricles and permits the return of normal cerebrospinal fluid flow there through.

### CROSS REFERENCE TO RELATED APPLICATIONS

This application claims the benefit under 35 U.S.C. §119(e) of U.S. Appl. No. 60/910,768 filed Apr. 9, 2007, which is incorporated by reference herein in its entirety.

### FIELD OF THE INVENTION

The invention relates generally to medical devices and methods for use during surgical procedures. More specifically, the invention is related to methods and apparatus for use in opening an obstruction within the brain.

### BACKGROUND OF THE INVENTION

Hydrocephalus is sometimes referred to as ‘water on the brain’. A watery fluid, known as cerebrospinal fluid or CSF, is produced continuously inside each of the four spaces or ventricles inside the brain. The CSF normally flows through narrow pathways from one ventricle to the next, then out across the outside of the brain and down the spinal cord. The CSF is absorbed into the bloodstream and recirculates. The amount and pressure of CSF in the brain are normally kept within a fairly narrow range. However, if the flow pathways are blocked at any point, the fluid accumulates in the cerebral ventricles and/or subarachnoid space, causing them to swell, which thereby results in an increase on intracranial pressure, and compression of the surrounding tissue. In babies and infants prior to the closure of the cranial sutures, hydrocephalus will cause the head to enlarge. In older children and adults, the head size cannot increase as the bones which form the skull are completely joined together and, as such, hydrocephalus may cause severe headaches, nausea, abnormal gait, dementia and/or permanent brain damage.

Non-communicating or obstructive hydrocephalus due to blood clots or other obstructions that restrict CSF flow within the ventricles or ventricular outlets, such as infectious material or tumors, is primarily treated by ventriculoperitoneal (VP) shunts. A shunt is a drain that diverts or “shunts” the accumulated CSF from the obstructed drainage pathways to another part of the body for absorption. These devices are catheters that are surgically lowered through a hole drilled in the skull and brain to have one end positioned in the lateral ventricle, while the other end of the catheter is tunneled under the skin and positioned in the peritoneal cavity of the abdomen, or right atrium of the heart, where the cerebrospinal fluid is absorbed or drained respectively. The catheter tubing has ports for receiving CSF that often get clogged by cellular in-growth and the catheters also utilize one-way valves or pressure valves. The failure rate for these devices ranges from 30% to 40% due to clogging of the catheter, infection, and/or faulty pressure or one-way valves. The surgical procedure requires burring holes in the skull and passage of the shunt through the cerebral cortex and underlying white matter, which may cause damage to those parts of the brain. VP shunts are also indicated for patients with communicating hydrocephalus, where CSF absorption into the venous system is restricted at the arachnoid granulations.

Recently neuroendoscopy, or telescopic surgery, makes treatment of hydrocephalus in some patients possible without shunting, the success rate depending on the etiology of the hydrocephalus. Management of hydrocephalus by endoscopic third ventriculostomy (ETV) involves creating an opening in the floor of the third ventricle, allowing the CSF to bypass the obstruction. This is a surgical procedure that does not have the complications of shunt insertion, i.e., infection is rare and morbidity is very low. However, ETV is not effective in patients with communicating hydrocephalus. Also, placement of a VP shunt and ETV procedures both require burr holes in the skull and introduction of medical apparatus through the cerebral cortex and underlying white matter.

What is needed are alternative treatment methods and apparatus that provide a less invasive approach to removing hydrocephalus causing obstructions within the brain and/or placing a shunt through the obstructed site to provide drainage of cerebrospinal fluid there through.

### BRIEF SUMMARY OF THE INVENTION

Embodiments hereof are directed to an intraventricular shunt for use in treating non-communicating hydrocephalus. The shunt includes a tubular structure having a length that substantially corresponds with a length of an opening in the brain in which the tubular structure is to be implanted, such as a length that corresponds with a length of one of the Foramen of Monro, cerebral aqueduct, Foramina of Magendie or Luschka, an arachnoid cyst, and a length for crossing the septum pellucidum. The tubular structure may be a polymeric cylindrical tube having the same diameter before and after implantation within the brain opening, or may have a fixed deployed diameter. In an embodiment, the shunt may have an hour glass shape or a single flared end in a deployed configuration. In another embodiment, the intraventricular shunt may be a self-expanding stent structure with a fixed expanded diameter.

Embodiments include methods for use within the subarachnoid space of the central nervous system to gain access to and treat an obstruction within the ventricles of the brain or a subarachnoid hemorrhage. A method of treating non-communicating hydrocephalus includes gaining access to the subarachnoid space of the spinal column and navigating an aspiration catheter through the subarachnoid space to the base of the skull. The aspiration catheter is then navigated along the brain in one of the subarachnoid space and ventricles until an obstruction for treatment is reached. A distal tip of the aspiration catheter is positioned proximal to the obstruction to be treated so that the obstruction may be aspirated and removed by the aspiration catheter. The aspiration catheter may be navigated into the fourth ventricle of the brain via one of the foramen of Magendie and the foramina of Luschka and subsequently through the ventricles of the brain until the obstruction is reached.

In another embodiment, a delivery catheter having a deployable intraventricular shunt thereon is navigated through the subarachnoid space to the base of the skull and enters the fourth ventricle of the brain via one of the foramen of Magendie and the foramina of Luschka. Navigation of the delivery catheter continues through the ventricles of the brain until the opening created by the aspirated obstruction is reached, and the distal end of the delivery catheter is positioned through the opening. An intraventricular shunt according to an embodiment hereof is then deployed within the opening and the delivery catheter is removed.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates portions of the anatomy of the brain.

FIG. 2 illustrates portions of the anatomy the lower spinal column with a catheter introducer and a catheter positioned within the subarachnoid space.

FIG. 3 illustrates a side view of an intraventricular shunt according to an embodiment of the present invention.

FIG. 4 illustrates a cross-section of an intraventricular shunt according to another embodiment of the present invention.

FIG. 5 illustrates a perspective view of an intraventricular shunt according to another embodiment of the present invention.

FIG. 6 illustrates a cross-section of an intraventricular shunt according to another embodiment of the present invention.

FIG. 7 illustrates a cross-section of an intraventricular shunt according to another embodiment of the present invention.

FIG. 8 illustrates a side view of an intraventricular shunt according to another embodiment of the present invention.

### DETAILED DESCRIPTION OF EMBODIMENTS OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

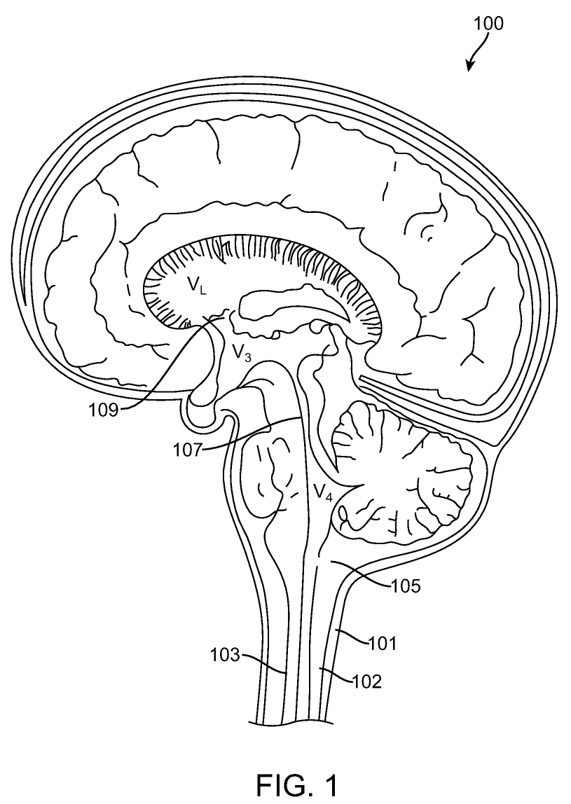

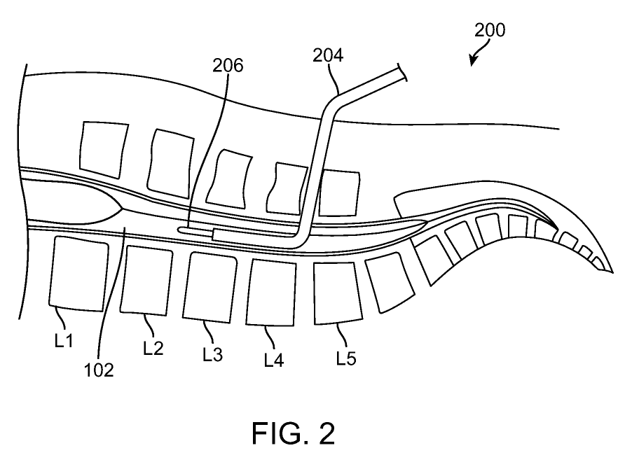

FIGS. 1 and 2 illustrate portions of the anatomy of the brain 100 and lower spinal column 200, respectively. Embodiments according to the present invention include medical apparatus and methods for breaking up and removing clots or other obstructions within the brain that cause hydrocephalus via access through the subarachnoid space 102. Subarachnoid space 102 is an area between the arachnoid mater 101 and pia mater 103 that surrounds the brain and the spinal cord and contains the cerebrospinal fluid. In an embodiment, an obstruction/clot within the ventricles of the brain may be accessed through the spinal subarachnoid space 102. Initially, a lumbar puncture may be performed at the L3-L4 or L4-L5 space or a cervical puncture, such that a catheter introducer 204, and/or a guiding catheter, may be positioned within subarachnoid space 102. A subarachnoid catheter 206, i.e., a steerable single lumen microcatheter, may then be inserted through catheter introducer 204 and into subarachnoid space 102. Subarachnoid catheter 206 may then be navigated to locations along the spinal cord surface or up to the skull base within subarachnoid space 102, where it may then be steered to areas on the brain surface, or through, for example, the foramen of Magendie 105 or one of the formina of Luschka (not shown) and into the fourth ventricle V₄. Subarachnoid catheter 206 may then be navigated cephalad through the cerebral aqueduct 107 and into the third ventricle V₃. If the obstruction is deeper within the ventricles, subarachnoid catheter 206 may be positioned through the interventricular foramen of Monro 109, as needed, to access one of the lateral ventricles V_{L}. The subarachnoid catheter can be navigated to the occlusion or hemorrhage site, for example, within the ventricles, interpeduncular cistern, etc. In addition, the subarachnoid catheter may be used to poke through or penetrate the septum pellucidum to treat asymmetric ventriculomegaly, drain an arachnoid cyst, and/or to allow drainage of excess CSF from the arachnoid cisterns.

Navigation of subarachnoid catheter 206 through subarachnoid space 102 and within the ventricles of the brain may be assisted with a guidewire. The position of the guidewire within the subarachnoid space and ventricles of the brain, as well as that of other medical devices used in accordance with methods herein, may be monitored by using any suitable imaging technology, such as magnetic resonance imaging, fluoroscopy, endoscopy, fiberoptic visualization, computed tomography, thermal imaging, sonography, X-ray visualization, and/or any combination of these. Accordingly, access to the ventricles via a lumbar or cervical puncture by methods according to the present invention significantly reduces recovery time to a day or sooner, instead of several days to weeks as is customary with the more invasive VP procedures currently in practice, which as mentioned may include burr holes through the skull and invasion of the cerebral cortex and subcortical white matter by medical apparatus.

If the non-communicating hydrocephalus is caused by a subarachnoid or intraventricular hemorrhage and clotting, or other obstruction, such as from infectious material, the obstruction may be slowly aspirated through subarachnoid catheter 206 using a controlled aspiration system to prevent excess removal of CSF and slit ventricle or collapsed ventricle situations. Removal of a subarachnoid hemorrhage will also reduce risk of severe vasospasm of the surrounding arteries and other SAH-induced adverse events. Once the obstruction is removed, aspiration or gradual shunting of CSF may continue until the excess cerebrospinal fluid within the ventricle is removed, with care being taken not to drain too quickly, or to remove too much cerebrospinal fluid and causing slit ventricle syndrome. The placement of subarachnoid catheter 206 and removal of cerebrospinal fluid may be monitored by fluoroscopy. A constant volume aspiration catheter as disclosed in provisional application U.S. Appl. No. 60/910,770 filed on Apr. 6, 2007, which is incorporated by reference herein in its entirety, may also be used to inject a fluid to break-up an obstruction while simultaneously aspirating the debris to maintain a constant volume aspiration.

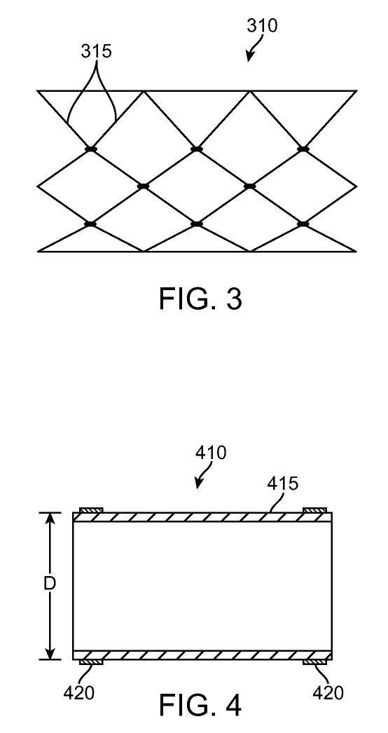

In another embodiment of the present invention shown in FIGS. 3 and 4, a subarachnoid delivery catheter may be used to deliver an intraventricular shunt 310, 410 to the ventricles via the subarachnoid space in a patient with non-communicating hydrocephalus. The delivery catheter is navigated across the obstruction that resisted complete aspiration by the subarachnoid aspiration catheter to deploy intraventricular shunt 310, 410 there through. Intraventricular shunt 310, 410 is positioned to reopen the pathway between the ventricles and permit the return of normal cerebrospinal fluid flow. Suitable delivery catheters adaptable for use with embodiments of the present invention include those disclosed in U.S. Pat. No. 5,833,694 to Poncet and U.S. Pat. No. 6,786,918 to Krivoruchko et al., each of which is incorporated by reference herein in its entirety.

Embodiments of intraventricular shunts according to the present invention are delivered to the treatment site by less invasive procedures than placement of a VP shunt or an ETV procedure making infection less likely. VP shunt valves for pressure relief and the excess catheter length thereof, which is required to accommodate growth in infants and children, are not needed with an intraventricular shunt according to the present invention, as it is sized to be positioned entirely within the treatment site in the brain.

In the embodiment of FIG. 3, intraventricular shunt 310 may include a self-expanding or balloon-expandable tubular stent structure 315. A self-expanding stent may be preferable for cases where high flexibility but lower radial force is needed upon implantation, whereas a balloon-expandable stent may be desirable for cases needing high radial force upon implantation, such as when placed proximate a brain tumor. Self-expanding stent structure 315 is constructed to radially expand from a compressed diameter into an expanded diameter against the walls of the opening within the obstruction to conform to the shape thereof. Once deployed, self-expanding stent structure 315 is radially pressed against the walls of the opening to remain in position, thereby minimizing post deployment displacement or shifting. Intraventricular shunt 310 may be made of a shape memory polymeric or metallic materials including nickel titanium alloys, such as nitinol (e.g., ELASTINITE® by Advanced Cardiovascular Systems, Inc., Santa Clara, Calif.) or other suitable material, examples of which include, but are not limited to, stainless steel, “MP35N,” “MP20N,”, tantalum, platinum-iridium alloy, gold, magnesium, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum, as well as resorbable and non-resorbable polymeric materials, such as PLA (polylactic acid), polycaprolactone, copolymers of lactic acid, glycolic acid, caprolactone, trimethylene carbonate, dioxanone in any permutation and combinations thereof.

In an embodiment, a stent design that minimizes herniation of brain parenchyma through the stent-like struts yet still allows access to and/or drainage of CSF may be beneficial. Stent structures having struts as shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau and U.S. Pat. No. 6,730,116 to Wolinsky et al., each of which is incorporated by reference herein in its entirety, may be adapted for use in embodiments of the present invention.

In another embodiment shown in FIG. 4, intraventricular shunt 410 may be used to maintain the patency of an opening previously closed by a tumor induced obstruction. Intraventricular shunt 410 is formed from a compliant or semi-compliant polymeric tubular structure 415 that is a solid-walled cylindrical tube having a diameter “D.” In an embodiment where shunt 410 may be used for temporary shunting, such as to open an edema-induced obstruction, shunt 410 may be made from a suitable bioresorbable polymer. In an embodiment, tubular structure 415 may have a “fixed” diameter, which means that shunt 410 has the same diameter in the deployed and undeployed state. In another embodiment, tubular structure 415 may be expandable, such as to be self-expanding or balloon expandable, to have a “fixed” deployed diameter, which means that shunt 410 does not expand beyond a certain deployed diameter such that no additional radial force is exerted on the surrounding tissue. As such, polymeric tubular structure 415 provides greater radial strength to intraventricular shunt 410 than would be provided by a self-expanding stent structure and is able to withstand increasing compressive forces, such as those caused by growing tumors adjacent to the ventricles or cerebral aqueduct. An additional benefit of using fixed-diameter polymeric tubular structure 415 within openings in the brain, for instance, within the foramen of Monro, is that when implanted tubular structure 415 does not provide any radial force onto the surrounding tissue, e.g., the choroid plexus of 3^{rd} ventricle, and therefore, does not interfere with the surrounding tissue or its function.

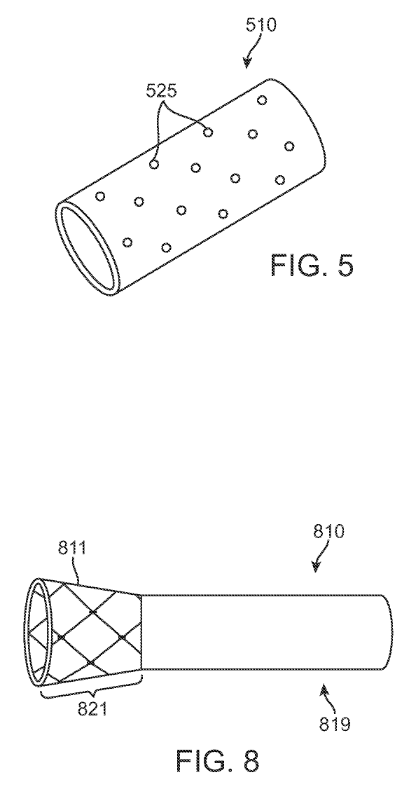

In another embodiment shown in FIG. 5, tubular shunt 510 may be formed from a polymeric tube having side ports 525 that allow the flow of CSF there through. Although ports 525 are shown somewhat evenly distributed along the length and about a circumference of shunt 510, ports 510 may be concentrated at one or both ends of the shunt and/or may be randomly spaced along the length and circumference thereof.

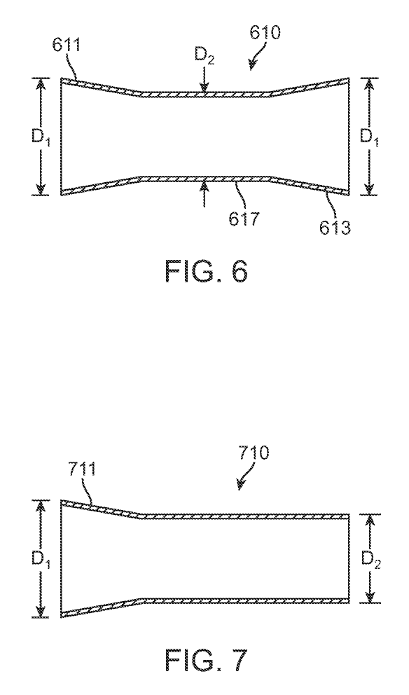

In another embodiment, a tubular shunt hereof may be a braided structure of a constant diameter, such as the embodiment of FIGS. 3-5, or have sections with varied diameters, as discussed in the embodiments of FIGS. 6-8 below. The braid density of the braided structure may be selected to provide the shunt with a certain property, e.g., a tight or high density braid may resist radial loads and provide better scaffolding in an opening subject to compressive forces. Alternatively, the braided structure may have distinct segments with different braid densities, such as a low braid density in a body segment to allow drainage through the sides of the shunt, in lieu of side ports 525, and a higher density in an end segment or segments where greater scaffolding is needed, such as for anchoring the device. A correctly sized braided shunt in accordance with an embodiment hereof that is placed in the cerebral aqueduct will not press against the brain, and will allow for some compression from the brain without completely closing. In such a situation where the CSF flow is normally low, placement of the braided shunt will be beneficial, even with a small or reduced inner diameter. A braided shunt may also be used at the foramen of Monro or Magendie.

In another embodiment shown in FIG. 6, tubular shunt 610 may be formed from a shape memory polymer or metal tube to be delivered to a treatment site within the brain, such as the cerebral aqueduct, in a cylindrical shape similar to FIG. 4 and thereafter to expand to an hour glass shape upon implantation, as shown in FIG. 6. Each of a proximal end 613 and a distal end 611 of hour-glass shaped shunt 610 expands to a diameter D₁ that is greater than a diameter D₂ of a midsection 617. Expanded diameter D₁ is sufficient to prevent migration of shunt 610 once the shunt has been implanted within, for instance, the cerebral aqueduct. In another embodiment, side ports, as shown in the embodiment of FIG. 5, may also be utilized in shunt 610 if shunt 610 is formed from a solid-wall polymeric or metallic tube.

In another embodiment shown in FIG. 7, tubular shunt 710 has a flared distal end 711. Flared distal end 711 has an expanded diameter of D₁ that is greater than a diameter D₂ of the remainder of shunt 710. As in previous embodiments, tubular shunt 710 may be made of a shape memory alloy or polymer to have an expanded/deployed configuration as shown in FIG. 7. Shunt 710 is delivered via a catheter to a treatment site, for instance, the cerebral aqueduct, in an unexpanded/undeployed configuration similar to the shape of shunt 410 in FIG. 4. Distal end 711 of shunt 710 is released from the catheter proximate the 3^{rd} ventricle such that distal end 711 regains its deployed/flared configuration. With distal end 710 flared in apposition with the cerebral aqueduct, shunt 710 is prevented from caudal migration toward or into the 4^{th} ventricle.

Alternatively, as shown in a side view of tubular shunt 810 in FIG. 8, distal end 811 may be formed of an expandable strut portion 821 that is attached to a tubular body portion 819. Tubular body portion 819 may be a constant diameter polymeric tube similar to previously described shunt 410. In various embodiments, expandable strut portion 821 may be of a shape memory material or other self-expanding structure, or may be selectively balloon expandable.

In an alternate embodiment, a tubular shunt in accordance herewith may include expandable proximal and distal segments, structurally similar to ends 611, 613 or 811 of the previous embodiments, that attach to each other at their non-flared ends without a constant diameter segment, such as midsection 617, therebetween.

Methods of delivery of shunts according to embodiments of the present invention will now be discussed with reference to the embodiment shown in FIG. 4. However, it should be understood by one of ordinary skill in the art that any of the intraventricular shunts disclosed herein may be delivered/implanted in a similar fashion. Intraventricular shunt 410 is deliverable through the subarachnoid space by a delivery catheter having a handle at its proximal end that provides accurate release of tubular structure 415, which is releasably attached to the delivery catheter's distal end. Intraventricular shunt 410 may include radiopaque markers 420 on its proximal and distal ends to aid in accurate placement of the shunt via fluoroscopy. Suitable delivery catheters adaptable for use with this embodiment of the present invention include those disclosed in U.S. Pat. No. 5,833,694 to Poncet and U.S. Pat. No. 6,786,918 to Krivoruchko et al, each of which is incorporated by reference herein in its entirety. In various embodiments of the present invention, shunt 410 may be a self-expanding prosthesis or as a balloon-expandable prosthesis having a deployed diameter that may be appropriately chosen by a clinician to be no more than 0.25 mm larger than the “natural” or native diameter of the lumen within which the prosthesis is to be placed, such that shunt 410 may be deliverable by any means known to one of ordinary skill in the art for delivering a self-expanding or balloon-expandable prosthesis. In an embodiment, a deployed diameter of the self-expanding or balloon-expandable prosthesis no longer exerts a radial force upon the deployment site and its surrounding tissue, such that the prosthesis may be consider “fixed” in diameter.

Polymeric tubular structure 415 may be made of one or more suitable polymeric materials, including a thermoplastic material, such as polyethylene block amide copolymer, polyvinyl chloride, polyethylene, polyethylene terephthalate, polyamide, polyurethane and/or a thermoset polymer, such as polyimide. In another embodiment of the present invention, tubular structure 415 may also be made of a resorbable polymeric materials, such as PLA (polylactic acid), polycaprolactone, copolymers of lactic acid, glycolic acid, caprolactone, trimethylene carbonate, dioxanone in any permutation and combinations thereof.

An intraventricular shunt according to embodiments of the present invention may be of varying diameters and lengths depending on the delivery site within the brain, i.e., whether the shunt will be delivered to or within the Foramen of Monro, 3^{rd} ventricle, cerebral aqueduct, 4^{th} ventricle, Foramina of Magendie or Luschka, arachnoid cyst, or across the septum pellucidum. In an exemplary embodiment, polymeric tubular structure 415 of intraventricular shunt 410 may be between 0.5 mm to 5 mm in diameter and up to 2 cm in length. More particularly, shunt 410 may be a length of between 2 mm and 4 mm to bridge the foramen of Monro or a length of between 10 mm and 16 mm to bridge the cerebral aqueduct.

Subarachnoid shunt delivery catheter may be tracked to the treatment site via a guidewire that has been previously positioned within the subarachnoid space to and beyond the obstruction. In certain medical applications, another subarachnoid catheter may already be in-dwelling, such that the guidewire may be tracked through the lumen of the in-dwelling subarachnoid catheter to and through the obstruction with the subarachnoid catheter being subsequently removed. Shunt delivery catheter may then be tracked along the guidewire up to the obstruction, with the guidewire being subsequently removed.

Subarachnoid delivery catheters for delivering intraventricular shunts in accordance with various embodiments of the present invention may be approximately 150 cm in length. A distal tip of a delivery catheter in accordance with various embodiments may include a rounded edge to minimize the likelihood of catching or tearing vessels, spinal nerve rootlets and central nervous system tissue as it is tracked to the cite of the obstruction within the brain ventricles. The distal tip of the delivery catheter may also include a radiopaque marker to facilitate accurate positioning of the catheter and the intraventricular shunt by fluoroscopy. In various embodiments, a distal portion of the delivery catheter may have a diameter ranging from 2 F to 9 F depending on the application in which it is to be used.

In a method according to an embodiment of the present invention, a clinician determines that a lumbar puncture may be performed without the risk of cerebral herniation. If cerebral herniation is of risk, burr holes can be made in the skull with a catheter placed in the adjacent subarachnoid space to relieve excess intracranial pressure. The lumbar puncture is performed with a catheter introducer, or another appropriate medical instrument, and a Touhy Borst valve is attached. An aspiration catheter is inserted through the Touhy Borst valve and catheter introducer to thereby gain access to the spinal subarachnoid space. The aspiration catheter is then navigated superiorly within the spinal subarachnoid space to the base of the skull. The aspiration catheter may then make entry into the fourth ventricle through the foramen of Magendie, one of the foramina of Luschka or navigated to the site of subarachnoid hemorrhage over the brain surfaces, i.e., cisterns at the skull base. The aspiration catheter is then tracked within the ventricles or subarachnoid space until the distal tip is positioned proximal to the obstruction. Radiopaque markers and/or fiber optic imaging may be used to aid in positioning of the distal tip of the catheter. Aspiration or attempted aspiration of the obstruction is performed and the aspiration catheter is withdrawn over the wire. If the obstruction cannot be aspirated, or there is a risk of re-occlusion, a shunt delivery catheter is then inserted through the Touhy Borst valve and introducer and navigated through the spinal subarachnoid space and/or ventricles of the brain to the site of the obstruction, as described above with reference to the subarachnoid aspiration catheter. The intraventricular shunt is then positioned across the obstruction using the radiopaque markers or fiberoptic visualization and released from the shunt delivery catheter. Once the shunt delivery catheter is removed, the aspiration catheter may be reintroduced to aspirate excess cerebrospinal fluid, if needed, and/or constant volume aspiration may be performed to clear debris in the CSF at the end of the procedure.

In another embodiment of the present invention, the clot or obstruction may be initially broken up or loosened by an ultrasonic medical device, such as any of the devices disclosed in U.S. Pat. No. 6,660,013 to Rabiner et al. and U.S. Pat. No. 6,652,547 to Rabiner et al., each of which is assigned to OmniSonics Medical Technologies, Inc. of Wilmington, Mass., or by another mechanical disruption provided by, for e.g., sinusoidal wires, coils and the like. The structure of a blood clot caused by subarachnoid or intraventricular hemorrhage may not necessarily form such that the clot is readily susceptible to aspiration. In some cases, blood clotting will occur along the ventricular walls making aspiration difficult. In addition, other types of obstructions that may cause hydrocephalus, such as infectious material and necrotic debris, may be difficult to aspirate without pretreatment. In such presentations where a clot or obstruction cannot be easily removed by aspiration alone, the clot or obstruction may be initially treated, i.e., loosened and/or broken up, through the use of a guidewire that generates ultrasonic waves in the cerebrospinal fluid, such as the ultrasonic medical devices disclosed in the OmniSonics patents mentioned above or by an ultrasound microcatheter such as those available from EKOS Corporation of Bothell, Wash. A method in accordance with an embodiment of the present invention, includes tracking an ultrasonic guidewire to the obstruction within the lumen of an in-place subarachnoid catheter and then activating the guidewire to break up the obstruction while the debris is aspirated through the catheter.

In another embodiment of the present invention, a blood clot may be treated by injecting rt-PA or other thrombolytics into the obstruction via the subarachnoid catheter, followed by aspiration. As previously discussed, the treatment of the blood clot with a constant volume aspiration catheter may be beneficial in improving aspiration efficacy. A constant volume aspiration provides injection of a fluid to break-up the obstruction with simultaneous aspiration of the debris while maintaining a constant volume of CSF.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. An intraventricular shunt for use in treating non-communicating hydrocephalus comprising:
a tubular structure having a length of between 2 mm and 16 mm that substantially corresponds with a length of an opening in the brain in which the tubular structure is to be implanted.

2. The intraventricular shunt of claim 1, wherein the tubular structure is a polymeric cylindrical tube having the same diameter before and after implantation within the brain opening.

3. The intraventricular shunt of claim 1, wherein the tubular structure is a polymeric cylindrical tube that when implanted in the brain opening has a fixed deployed diameter.

4. The intraventricular shunt of claim 1, wherein the tubular structure is a polymeric tube having an hour glass shape in a deployed configuration.

5. The intraventricular shunt of claim 1, wherein the tubular structure has a flared end in a deployed configuration.

6. The intraventricular shunt of claim 5, wherein the flared end includes a strut portion that is expandable upon deployment.

7. The intraventricular shunt of claim 6, wherein the expandable strut portion is attached to a tubular body portion that includes a constant diameter polymeric tube.

8. The intraventricular shunt of claim 7, wherein the expandable strut portion is of nitinol.

9. The intraventricular shunt of claim 1, wherein the tubular structure is a self-expanding tubular stent structure having a compressed diameter and a fixed expanded diameter.

10. A method of treating non-communicating hydrocephalus, comprising the steps of:
providing an aspiration catheter;
gaining access to the subarachnoid space of the spinal column;
navigating the aspiration catheter through the subarachnoid space to the base of the skull;
navigating the aspiration catheter along the brain in one of the subarachnoid space and ventricles until an obstruction for treatment is reached;
positioning a distal tip of the aspiration catheter proximal to the obstruction to be treated; and
aspirating the obstruction and removing the aspiration catheter.

11. The method of claim 10, wherein the steps of navigating the aspiration catheter include entering the fourth ventricle of the brain via one of the foramen of Magendie and the foramina of Luschka and navigating through the ventricles of the brain until the obstruction is reached.

12. The method of claim 10, wherein the step of aspirating the obstruction includes one of creating an opening through the obstruction and creating an opening by removing the obstruction.

13. The method of claim 12, further comprising:
providing a delivery catheter having a deployable intraventricular shunt at a distal end thereof;
accessing the subarachnoid space with the delivery catheter;
navigating the delivery catheter through the subarachnoid space to the base of the skull and entering the fourth ventricle of the brain via one of the foramen of Magendie and the foramina of Luschka;
navigating the delivery catheter through the ventricles of the brain until the opening created by the aspirated obstruction is reached;
positioning the distal end of the delivery catheter through the opening; and
deploying the intraventricular shunt within the opening and removing the delivery catheter.

14. The method of claim 10, wherein the steps of navigating the aspiration catheter include navigating the aspiration catheter in the subarachnoid space within the brain to a hemorrhage site, wherein the hemorrhage site is the obstruction to be treated.

15. The method of claim 14, further comprising:
providing a delivery catheter having a deployable intraventricular shunt at a distal end thereof;
accessing the subarachnoid space with the delivery catheter;
navigating the delivery catheter through the subarachnoid space to the base of the skull and along the brain;
navigating the delivery catheter through the subarachnoid space of the brain until the aspirated obstruction is reached;
positioning the distal end of the delivery catheter through the obstruction; and
deploying the intraventricular shunt within the obstruction and removing the delivery catheter.

16. The method of claims 13 and 15, wherein the intraventricular shunt includes a polymeric cylindrical tube having the same diameter before and after deployment within the brain.

17. The method of claims 13 and 15, wherein the intraventricular shunt has a fixed deployed diameter and is of a stent structure selected from the group consisting of a self-expanding stent structure and a balloon-expandable stent structure.

18. The method of claims 13 and 15, wherein the intraventricular shunt includes a polymeric cylindrical tube having an hour-glass shape in a deployed configuration.

19. The method of claims 10, 13 and 15, wherein the step of gaining access to the subarachnoid space includes a lumbar puncture between one of the L3 and L4 vertebrae and the L4 and L5 vertebrae.

20. The method of claims 10, 13 and 15, wherein the step of gaining access to the subarachnoid space includes a cervical puncture.

21. The method of claims 13 and 15, wherein the intraventricular shunt includes a radiopaque marker and the step of positioning the distal end of the delivery catheter is aided by fluoroscopy.

22. The method of claims 13 and 15, wherein the step of positioning the distal end of the delivery catheter is aided by visualization via fiber optic navigation.

23. The method of claim 13, wherein the step of navigating the delivery catheter through the ventricles includes passing the delivery catheter through the fourth ventricle and the cerebral aqueduct and accessing the third ventricle.

24. The method of claim 13, wherein the step of navigating the delivery catheter through the ventricles further includes passing the delivery catheter through the foramen of Monro and accessing one of the left or right lateral ventricle.

25. The method of claim 13, wherein the step of navigating the delivery catheter through the ventricles further includes passing the delivery catheter through the septum pellucidum and accessing the contralateral lateral ventricle.

26. The method of claim 13, wherein the step of navigating the delivery catheter through the ventricles includes poking a hole in the floor of the third ventricle for directly accessing the third ventricle.
